(19) United States
(12) Patent Application Publication
(10) Pub. No.: US 2024/0148260 A1
(43) Pub. Date: May 9, 2024

(54) System and Method for Monitoring of Humans for Medical Purposes

(71) Applicant: REST Technologies, Inc., Sugar Land, TX (US)

(72) Inventor: Jerald H. Simmons, Bellaire, TX (US)

(73) Assignee: REST Technologies, Inc., Sugar Land, TX (US)

(21) Appl. No.: 18/503,685

(22) Filed: Nov. 7, 2023

Related U.S. Application Data
(60) Provisional application No. 63/383,056, filed on Nov. 9, 2022.

(51) Int. Cl. A61B 5/0205; A61B 5/00; G16H 20/00; G16H 80/00

(52) U.S. Cl. CPC A61B 5/0205; A61B 5/0022; A61B 5/4836; A61B 5/4848; A61B 5/6801; A61B 5/7435; A61B 5/7465; G16H 20/00; G16H 80/00; A61B 5/021

(57) ABSTRACT

A system and method for monitoring humans for medical purposes is provided. One method employs a communication network, the communication network including a plurality of network equipment. The method includes monitoring a physiologic event of a patient, then also obtaining other data from the patient, such as sleep data. The data is then displayed on the plurality of network equipment and also transmitted through the communication network so that at least two remotely located care givers can view the sleep data and the physiologic event on the plurality of network equipment. This enables the care givers to adjust a therapy for the patient based on the physiologic event and the other data.

### REFERENCE TO RELATED APPLICATION

Priority is claimed to provisional application Ser. No. 63/383,056, filed Nov. 9, 2022, entitled: “System and Method for Monitoring of Humans for Medical Purposes,” which is referred to and incorporated herein in its entirety by this reference.

### FIELD OF THE INVENTION

The present invention generally relates to monitoring of humans for medical purposes.

### BRIEF DESCRIPTION OF THE DRAWINGS

Various embodiments of the present invention taught herein are illustrated by way of example, and not by way of limitation, in the figures of the accompanying drawings, in which:

FIG. 1 illustrates an embodiment of the present invention that employs a communication network where aspects of the present invention may be deployed;

FIG. 2 illustrates a display on a portable electronic device according to one embodiment of the present invention;

FIG. 3 illustrates a second display on a portable electronic device according to one embodiment of the present invention;

FIG. 3A illustrates a third display on a portable electronic device according to one embodiment of the present invention;

FIG. 4 illustrates a fourth display on a portable electronic device according to one embodiment of the present invention;

FIG. 5 illustrates several graphed physiologic outputs and a timeline displayed on an electronic device according to one embodiment of the present invention;

FIG. 5A illustrates a close-up of a portion of the graphed physiologic outputs and the timeline as shown in FIG. 5;

FIG. 6 illustrates other graphed physiologic outputs displayed on an electronic device according to one embodiment of the present invention;

FIG. 7 illustrates yet other graphed physiologic outputs displayed on an electronic device according to one embodiment of the present invention;

FIG. 8 illustrates a display on an electronic device according to another embodiment of the present invention; and

FIG. 8A illustrates a display on an electronic device related to the embodiment illustrated in FIG. 8.

It will be recognized that some or all of the figures are schematic representations for purposes of illustration and do not necessarily depict the actual relative sizes or locations of the elements shown. The Figures are provided for the purpose of illustrating one or more embodiments of the invention with the explicit understanding that they will not be used to limit the scope or the meaning of the claims.

### DETAILED DESCRIPTION OF THE PREFERRED EMBODIMENTS

In the following description, for the purposes of explanation, numerous specific details are set forth in order to provide a thorough understanding of a medical monitoring system that embodies principals of the present invention. It will be apparent, however, to one skilled in the art that the medical monitoring system may be practiced without some of these specific details. Throughout this description, the embodiments and examples shown should be considered as exemplars, rather than as limitations on the medical monitoring system. That is, the following description provides examples, primarily using sleep monitoring as a use case, and the accompanying drawings show various examples for the purposes of illustration. However, these examples should not be construed in a limiting sense as they are merely intended to provide examples of the medical monitoring system rather than to provide an exhaustive list of all possible implementations of the medical monitoring system.

Specific embodiments of the invention will now be further described by the following, non-limiting examples which will serve to illustrate various features. The examples are intended merely to facilitate an understanding of ways in which the invention may be practiced and to further enable those of skill in the art to practice the invention. Accordingly, the examples should not be construed as limiting the scope of the invention. Other applications beyond sleep monitoring are within the scope of the medical monitoring system. In addition, reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner in one or more embodiments.

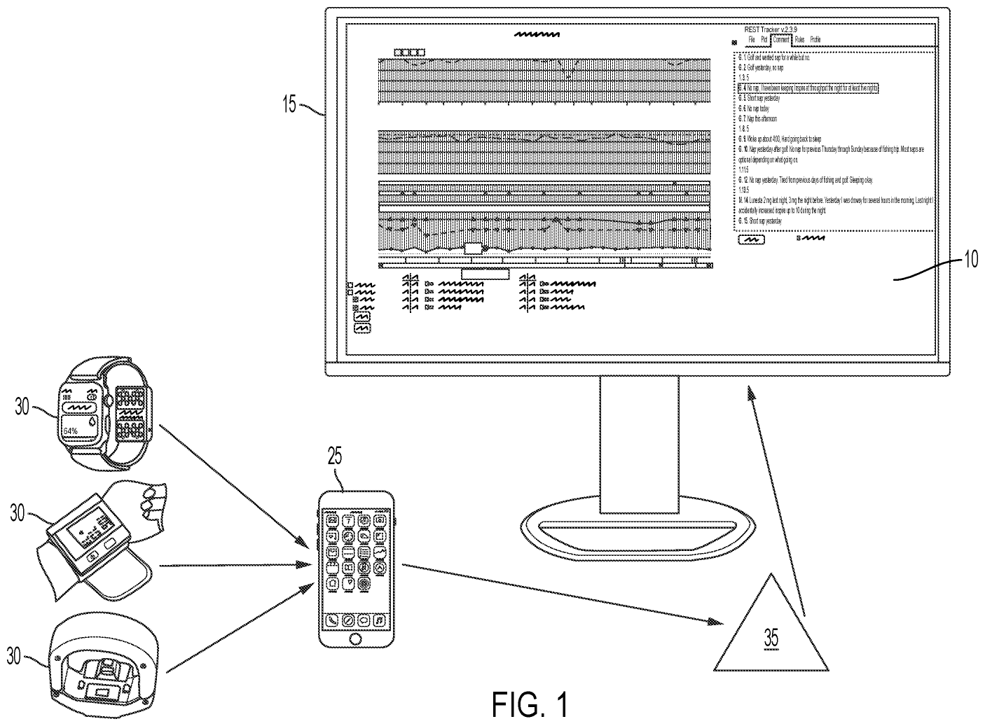

As illustrated and discussed in FIGS. 1-8A, the present invention comprises a system, method and computer program product, or computer software that provides a way to bring together multiple types of wearable sensor technology with patient inputs in a fashion that allows for multiple parameters to be brought together and displayed coherently. Embodiments of the present invention comprise computer software in the form of an computer application for a portable electronic device, such as a cell phone or electronic tablet. In addition, another embodiment of the present invention includes computer software for a desktop computer or computer server. The present invention functions as an integration hub, which allows interchangeable components used in medical monitoring. Features of the system are those needed to provide functionality to a medical team using the system to monitor a large number of patients.

One embodiment of the present invention is used for monitoring of sleep. However, other embodiments of the present invention may be employed for any type of remote patient monitoring. The present invention provides a computer program product, or computer software that works as a backbone for many types of physiologic monitoring devices. The present invention is designed to assist a clinical practice to monitor a large number of patients and also enables patient billing using new procedure codes. The codes have specific rules that must be followed and the present invention enables a clinical practice to abide by these rules.

For example, in a preferred embodiment the present invention provides a computer program product for healthcare providers to collaborate in a patient's case by ongoing monitoring of the patient that includes a time line with various physiologic parameters included. The computer program product permits annotations to be made by patients, the main monitoring physician, and collaborating clinicians and allows all of the collaborating clinicians to view all the data and pertinent notes from each of the collaborating clinicians. The data is labeled in a ledger format where each clinician has a unique identifier as does the patient, where the use of the computer program product is independent from a particular clinician's medical record system. The collaborative features of the present invention enable multiple care givers, or clinicians, that are co-located, or remotely located anywhere in the world, to have their analysis captured, stored and viewed by other clinicians. This collaborative feature improves the patient's quality of care or treatment by enabling multiple clinicians to analyze a patient's data and suggest treatment options. For example, each clinician can enter a comment, or annotation that is distinguishable on a timeline, as each clinician is represented by their own unique icon.

The present invention allows a platform for collaborative care not currently available, where the main element bringing the clinicians together is the monitoring of the patient, as opposed to current frameworks where the main element is the patient in a static format with no clear timeline structure, with clusters of disconnected elements held within each clinician's independent medical record system. In the present invention, each annotation has a place in the timeline of the monitoring of the patient.

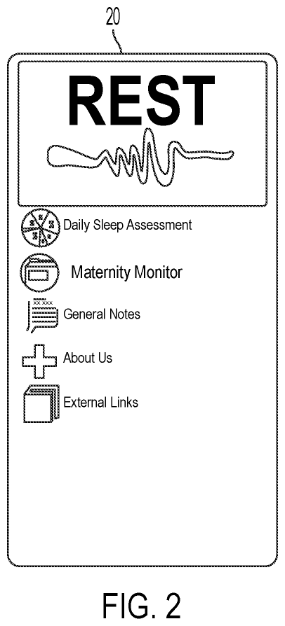

Referring now to FIG. 1, the present invention comprises a computer program product, or desktop software (called “REST desktop”) 10 that presents an integrated clinical data display on a desktop computer monitor 15 for remote patient monitoring, bringing data relating to a patient's sleep together with any patient data from a “REST” software application, or mobile application 20, shown in FIG. 2. As illustrated in FIG. 1, the REST software application, or REST mobile app 20, is programmed or downloaded into a cell phone, electronic tablet, or other electronic device 25. The REST mobile app 20 can receive and display data from many types of wearable technology. For example, as show in FIG. 1, the REST mobile app 20 can receive, via a wireless connection such as Bluetooth, and display, data from a variety of wearable technology 30, including a wrist watch, wrist strap, blood pressure monitors, chest straps, and other types of wearable medical devices for display on the REST desktop 10 data display and also on the REST mobile app 20. Specific examples of other types of wearable technology 30 that may be employed include: cardiopulmonary coupling technology via a Ring Oximeter such as a SleepImage System product that performs cardiopulmonary coupling analysis with a variety of output metrics; oxygen and heart rate from the Wellue, or Oura ring products; oral appliances that provide head position, snoring and oxygen levels; ankle bands with an accelerometer measuring leg movements; and a head cap that measures brain wave activity. The REST desktop 10 and REST mobile app 20 can also receive data from positive airway pressure (PAP) devices, such as is continuous positive airway pressure (CPAP) or BiLevel PAP from devices such as the ResMed line of CPAP machines, or the AirMini CPAP machines, or other CPAP machines, which are used to treat Obstructive Sleep Apnea (OSA), or negative pressure devices such as iNAP In other embodiments, a dental appliance that provides an output of compliance or mandibular position can be used in the REST software, for treatment of OSA. Other treatments such as the amount of insulin delivered by an insulin pump for treatment of diabetes are also contemplated, allowing as inout any drug infusion device that can be connected to the REST software. This data can be viewed along with the other data on the same screen in the REST desktop software 10.

In addition, embodiments of the present invention can receive and display data from other technology not related to human sleep. For example, other devices that may be employed by the present invention include: a scale for body weights over time; blood pressure monitoring devices; Dexcom glucose monitoring patches and body temperature devices. In addition, embodiments of the present invention can receive and display data from a finger ring to measure oximetry, heart rate, blood pressure, skin temperature, galvanic skin response or electrodermal skin response; motion via accelerometers or some other form of position monitoring; devices that track skin inductance changes, glucose level, CO2 level and lactic acid level. In addition, embodiments of the present invention can receive and display data from wrist devices or a belt that measures all the same parameters as listed for the finger ring. Also, embodiments of the present invention can receive and display data from a patch placed on the skin area such as forehead, chest, extremities that measure all the parameters as stated above plus electrical responses such as an ECG. In addition, embodiments of the present invention can receive and display data from a dental appliance the measures many of the parameters listed above plus head position, occlusal pressure (from biting down), mandibular position and snoring. In addition, embodiments of the present invention can receive and display data from a probe placed on the face area, such as the chin that measures many of the parameters listed above in connection with the dental appliance. In addition, embodiments of the present invention can receive and display data from: a probe placed on or under the undergarments to monitor for leakage of urine or stool; a probe to measure the level of tumescence (erection) of a male patient; a probe that measures blood flow by any means, that is portable and can be worn, over any area. In addition, embodiments of the present invention can receive and display data from a device that measures mobility such as the number of flexion or extension events at a joint, such as the elbow and the degree of such flection or extension movements. For example, this would include a sensor that measures head movements during a rehab process following neck surgery. In addition, embodiments of the present invention can receive and display data from activity monitoring devices using accelerometers worn on any location that can provide a metric of mobility to be quantified such as walking, running, and/or standing. In addition, embodiments of the present invention can receive and display data from a strap or patch or some other probe that would tabulate the frequency of limb muscle contractions of specific muscles or just a general limb movement, such as those that occur in patients with periodic leg movements disorder of sleep. In addition, embodiments of the present invention can receive and display data from devices that measure eye movements from either a specialized patch around the eye or via specialized glasses, such that eye movement functionality can be monitored throughout day or night, as would be important in conditions such as concussion recovery, or progressive neurologic disorders or vestibular abnormalities. In addition, embodiments of the present invention can receive and display data from a cap or other apparatus placed on the head that provides measurement of electroencephalographic signals from the brain.

As shown in FIG. 1, the present invention comprises a software application, the REST mobile 20 that can be used by a patient on a cell phone or electronic tablet or another device 25. Data received from the wearable technology 30 and information input by the patient on the REST mobile app 20 is sent via a network 35 to the REST desktop software 10. In an alternative embodiment, the wearable technology 30 may bypass the REST mobile app 20 and send data from the wearable technology 30 directly to the network 35. The network 35 is described in detail below and includes any one or both of a voice call and data communication using a public telephone network, in addition to communication using a wired or wireless TCP/IP protocols including Gigabit Ethernet, 4G, and 5G and future G6. The REST desktop software 10 then displays the data received from the REST mobile app 20 on a computer monitor 15. It will be appreciated that the network 35 can transmit the patient's data to a plurality of network equipment, or computer monitors 15 that have the REST desktop software 10 so that a plurality of care givers, doctors or other clinicians and medical staff can view the patient's data, either synchronously or asynchronously, in real time, or at a time of the user's choosing.

The present invention can assist physicians in providing care in a more meticulous fashion with an improved outcome. One embodiment of the invention as an example focusses on sleep and sleep disorders. However, beyond those conditions, the present invention can be adapted to serve other clinic conditions such as CHF (congestive heart failure), renal failure, and other medical conditions. The present invention integrates data from physiologic monitoring into the same display as patient input of symptoms, which allows for a superior method of demonstrating cause and effect and other relationships such as treatment responses to medications.

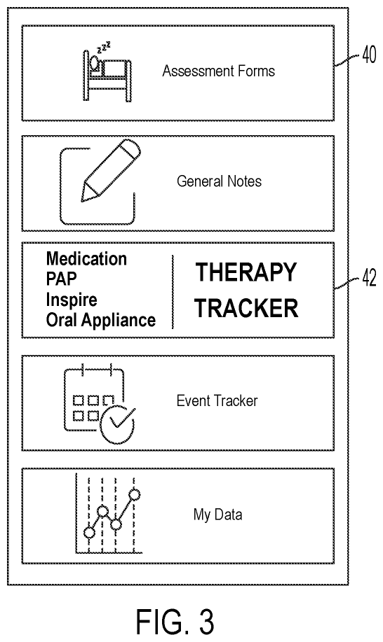

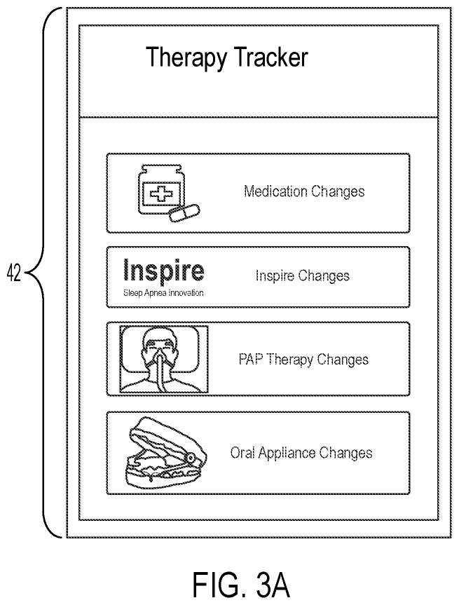

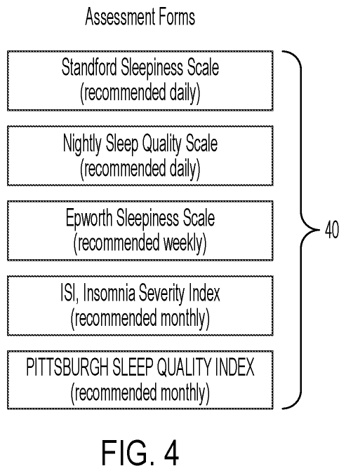

As illustrated in the FIGS. 2-4, one embodiment of the REST mobile app 20 allows for entries of patient information. Also, the REST mobile app 20 can be configured to allow different entries for different types of medical issues. For example, FIG. 2 includes a maternity monitor for pregnant patients, and FIG. 3 shows the REST mobile app 20 configured for general sleep monitoring. As shown in FIG. 3, the REST mobile app 20 includes assessment forms 40 and other information for other types of entries. FIG. 4 shows the assessment forms available for completion by the patient. Additionally, the application allows patients to view their own sleep data. They can select from a list of variables to view their data in a variety of time frames. The REST mobile app 20 also allows patients can list events such as seizures or headaches, so these events can be tracked, and a data displayed in the REST desktop 10 for review, eliminating the need for the patient to keep an independent log of such clinical events. Additionally, data can be displayed on the REST mobile app 20 for their review, such as the cardiopulmonary coupling data collected from the Sleep Image Ring. The clinician can select what data can be made available uniquely to each patient. For example, the present invention enables the nightly tracking of patients to assess therapeutic interventions and monitor their condition. This includes nightly cardiopulmonary coupling via a Ring Oximeter. Cardiopulmonary coupling analysis provides an output of total sleep time. For example, there are instances where a clinician may choose not to allow the patient to view an output because of concern that it may add to the anxiety of the patient, such as in the case of a patient who may also has obsessive compulsive disorder. Therefore, limiting the patient's access to that specific parameter may be of clinical importance. The ability to customize the viewable data for patients allows the clinician choices to maintain the best clinical care for a patient by modifying the access to their data. Accessibility can be modified as deemed clinically relevant by the treating clinician. Multiple medical wearable devices may be employed by the present invention, with each device acquiring data that can be integrated and displayed on the REST desktop 10.

As an example of other medical conditions that the REST desktop 10 and REST mobile app 20 can be used to monitor, seizure activity could be monitored in patients with epilepsy. There are new electroencephalography (EEG) technologies that have become available that allow a patient to wear a head cap which incorporates electrodes to record brain wave EEG activity without the necessity of prepping the skin or use of electrode gels. This allows the patient to simply place the cap on which will record the EEG data. One such system is the DREEM headband and another is the Muse. Both systems record brain wave activity. This data can be recorded by the REST mobile app 20 and transmitted to the REST desktop 10, and a patient can be monitored for seizures as well as relationships between factors such as sleep hygiene and how this may impact a patient's seizure frequency.

Another example would be for patients that suffer from irritable bowel syndrome (IBS). There are devices such as the Aire 2 by FoodMarble that is a small breath analyzer that determines the amount of methane and hydrogen in a person's breath. These gases relate to digestion of foods and have been shown to provide information related to IBS symptoms. This allows a patient to modify their food intake based on the influences it has on the production of these gases. This information can be recorded by the REST mobile app 20 and transmitted to the REST desktop 10, with other inputs such as sleep, seizures or even cardiac activity, if also monitored.

Another example of a clinical condition in which the REST mobile app 20 can be used would be for patients with intermittent atrial fibrillation using a long-term heart monitor such as the Philips MCOT system that measures heart activity. This data can be interfaced similarly to other data sets and incorporated into the comprehensive REST desktop 10 such that instances of atrial fibrillation can be linked to symptoms or related to food intake from the Aire 2 or sleep abnormalities identified by the SleepImage Ring or seizure activity collected from the DREEM EEG head band.

In an embodiment of the present invention used for analysis of sleep disorders, the invention includes sleep monitoring development and implementation. In this embodiment, to perform remote patient monitoring, the invention starts with a sensor on the patient's body, ranging from oximetry, body temperature, heart rate etc. The dataset obtained from the wearable device 30 is transmitted to the REST mobile app 20 and then transmitted to the REST desktop 10 via the network 35. Alternatively, the wearable device 30 may transmit data directly to the REST desktop 10. For example, in a system designed to assist in sleep disorders medicine, the following may be considered: a symptoms log; a medication log that can be organized for a range of conditions. Specific categorization of entries get placed into a catalog that allows for cross referencing. An example of how this could be used would be to select insomnia when doing a symptom retrieval search from collected data. This could assist in the specific tracking of the patients' behaviors from and effects on sleep, providing better insight into the treatment of insomnia. Also a log of changes relating to the use of PAP treatment by switching between different types of CPAP masks from one night to the next. Also, a log of changes relating to the use of Inspire HGNS therapy and objective data from a patients Inspire HGNS. Also, a log of changes relating to the use of a dental appliance designed for the treatment of OSA such as the level of advancement of the lower portion of the appliance which brings the mandible forward and opens the airway.

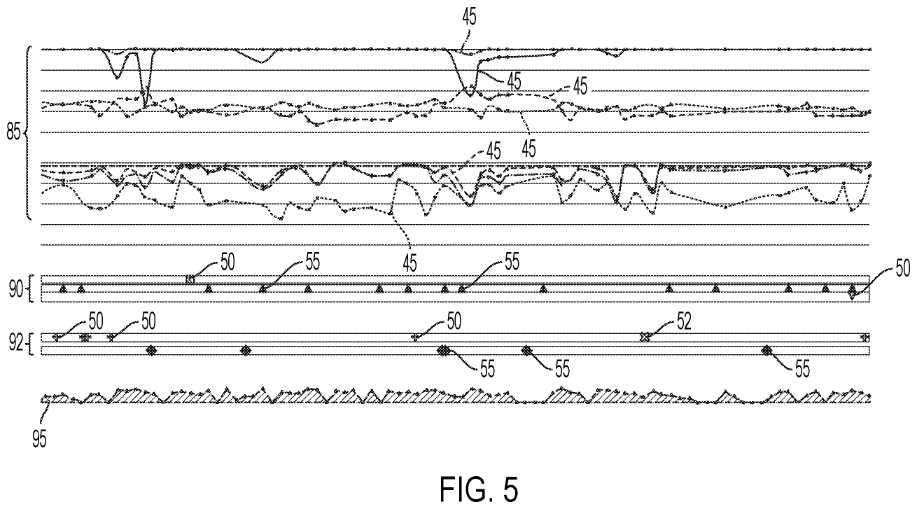

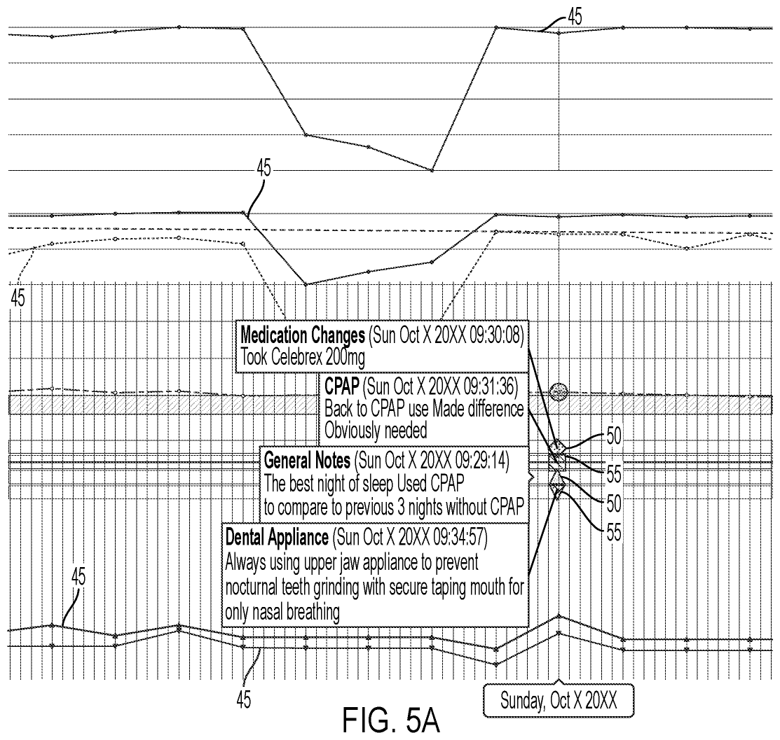

Referring now to FIGS. 5 and 5A, a portion of the REST desktop software 10 is illustrated. Several graphed physiologic outputs, or calculated physiologic outputs 45 are shown, with the data used to generate a graph, or display received from the REST mobile app 20, sleep study data (i.e., data received from a SleepImage System Ring device, for example) and from other sources, as listed herein. A adjustable timeline 95 allows a clinician to increase or decrease the amount of time displayed. Shown at the bottom of FIG. 5 is the recording timeline 95 that displays the recording time for each night. For example, the recording timeline 95 may display CPC SleepImage Ring data, but may also display other data types in which long segments of time are collected to provide an output versus a continuous 24 hour type of monitoring (such as glucose). If there is no data for that night then it is displayed in red or the timeline goes to the bottom or zero. As shown in FIG. 5, and in close-up in FIG. 5A, are triangular icons 50 and square icons 55 that are examples of icons from different categories that comprise a patient's comments or annotations and may be different colors and displayed in different vertical positions, each representing a different category, with the entry marked by the colored, shaped, icon on the appropriate vertical position along the timeline. Each icon may include information from one or more clinicians or the patient and different colors may represent different categories. For example, when a cursor selects a green triangle, a general comment will appear. When a cursor selects a red triangle, information on an oral appliance will appear.

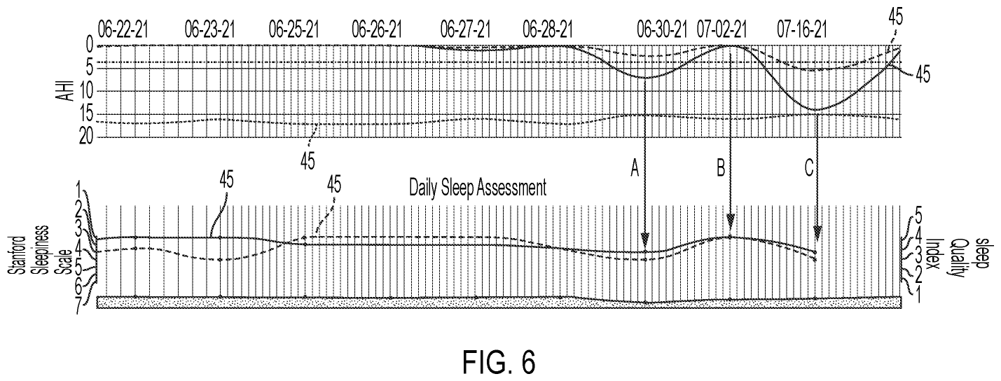

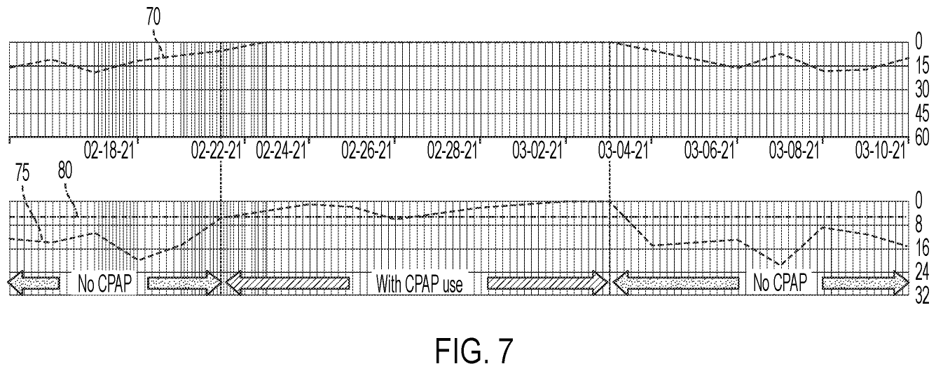

As shown in FIG. 6, the graphed physiologic outputs, or calculated physiologic outputs may represent data from any number of data sources. For example, FIG. 6 shows a display of several outputs linked to the patients daily sleep assessment scores. Responses on both the Stanford Sleepiness Scale and Sleep Quality Index are lower following nights with a higher Apnea Hypopnea Index (AHI), as shown by the A and C arrows. Other physiologic data from the patient may be displayed, such as: the total sleep time; amount of time oxygen was below 90%; the amount of time the patient used CPAP; the average pressure the CPAP administered each night as the total number of respiratory events per hour of sleep; the oxygen saturation as measured by blood analysis; an obstructive sleep apnea event; an oxygen desaturation; a percent oxygen saturation; data from a continuous positive airway pressure (CPAP) device, and other data. For example, as shown in FIG. 7, a patient with poor CPAP compliance from discomfort, is monitored nightly with data sent to the REST mobile app 20. That data is transmitted to the REST desktop 10 and displayed as graphed physiologic outputs. The upper graphed physiologic output 70 shows a percentage of sleep time with oxygen saturation below 88%. A lower graphed physiologic output 75 shows an Apnea Hypopnea Index (AHI), compared with a threshold of abnormal values 80. These are synchronous graphed physiologic outputs used to compare a patient's condition when using, and not using a CPAP device.

Now referring back to FIG. 5 that shows several graphed physiologic outputs, or calculated physiologic outputs 45. The upper group 85 can be a display of physiologic data such as those from the SleepImage Ring. The middle group 90 may be a display of inputs from the patient, such as symptoms or changes in treatments. The inputs may come from the patient, from the Therapy Tracker feature, discussed below. The lower group 92 may be a display of icons including the clinician annotation icon. The bottom line 95 is a display of the timeline that includes the time of day that the patient initiates nightly monitoring (or “bedtime” in the case where the wearable is continuously recording the patient and not only at night). For example, the time of day that sleep onset occurs and the time in which end of sleep occurs. These features are for the assessment and monitoring of patients in whom there is a concern of insomnia. It will be appreciated that different embodiments of the REST desktop software 10 can be configured to change the data that is displayed. For example, in one embodiment of the REST desktop software 10, illustrated in FIG. 5, the middle group 90 may represent patient annotations 55 with each horizontal line representing a different category from the therapy tracker (oral appliance, PAP, medication, etc.) and each horizontal line (i.e., category) may have its own unique icon to make each icon easier to distinguish. In this embodiment, and as shown in FIG. 5, the lower group 92 may show clinician annotations 50 and collaborating clinician annotations 52 in the first horizontal line, with the second horizontal line having diamond shaped icons 55 representing staff comments who have reached out to the patient to perform synchronous communication events. By scrolling over each icon, 50, 52 or 55, any notes entered by the representative individual will be displayed.

The REST desktop software 10 also includes a display of all of the patient inputs (comments) list chronologically for those inputs that show up adjacent to the graphed physiologic outputs 45. The REST desktop software 10 can also display a list of physiologic variables listed on the graph, each of which can be individually selected to be on or off. Also displayed is an average and standard deviation. Also, a clinician set the beginning and ending dates of the display window by selecting specific dates. As shown in FIG. 5, the icons 50 and 55 can be selected and as the cursor is scrolled over the different inputs from the patient, the comments entered pop up. Each category of inputs has an specific icon, as discussed above. In addition, the scale of each graphed physiologic output 45 can be adjusted as well as reversed. Importantly, as mentioned above, the REST desktop software 10 can display a patient's data to multiple clinicians simultaneously so that collaboration among the clinicians can result in quicker and better treatment options for the patient.

The present invention also allows a user, or clinician to save the display parameters into a “Template” so that the exact configuration can be pulled up again. A user can upload from previously saved Templates. Whenever making a change to the current display features, it changes the color of the template name to alert the user that there is a change in the display that deviates from the loaded template. If the user selects “save” then it will write over the template parameters with the new settings. However, if the user changes the name of the template it will save under the new name, keeping the previously template unchanged. The REST desktop software 10 also includes templates with pre-set features for the graphs. These templates are named. The user can quickly switch between templates when viewing data. For example, one template can focus on parameters specific to sleep apnea and another template can focus on parameters specific to insomnia and another may be for diabetes glucose monitoring. This provides a unique environment to appreciate relationships between different types of factors such as sleep apnea and diabetic control. The data is obtained from the REST mobile app 20, and other test data and are collected in a fashion that is organized into several categories, such as: Daily Assessment Scales that include sleepiness scales, sleep quality scales and other symptom scales. Also there is a Therapy Tracker that includes a medication tracker, inspire therapy notes, PAP therapy notes and oral appliances notes. Also there is an Event Tracker that includes seizures, headaches and other spells. Finally, there are General Notes for entries by the patient with comments that do not fit into any of the above sections. There are also different categories of inputs for the REST desktop software 10 that can be configured. For example, in a case of sleep medicine using the CPC Ring acquisition device and analysis to track sleep, the therapy tracker acquires data from appropriate sources, such as CPAP machines for patients on CPAP treatment, or Inspire HGNS devices for patients on Inspire HGNS devices. The patient can also input comments relevant to a specific mode of therapy. An example would be for a patient in CPAP therapy, so that the patient can place a comment when they switch from one type of CPAP machine to another. This allows the clinician to see if this switch makes a difference in their treatment by labeling when this action took place. Additional features of the present invention may include, allowing the clinician to add time sensitive comments adjacent to the graphed physiologic outputs 45. For example, the clinician can add comments that are date and time stamped and displayed as an icon adjacent to the graphed physiologic outputs 45. This would allow the clinician to edit patient's inputs such that the original input is saves but the clinicians comments annotate or replace the patients note.

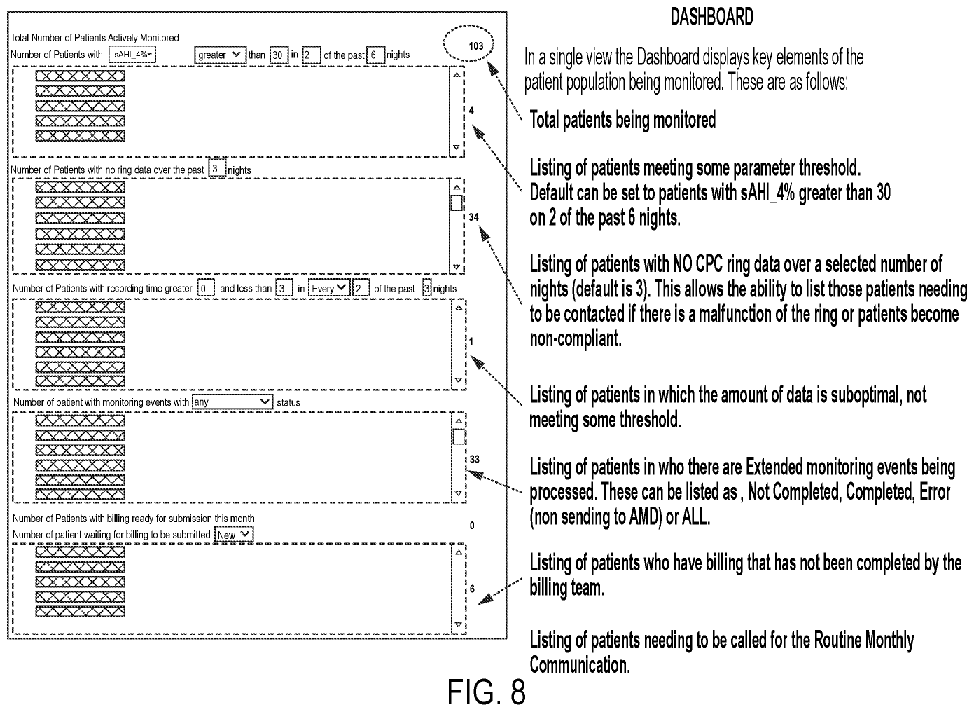

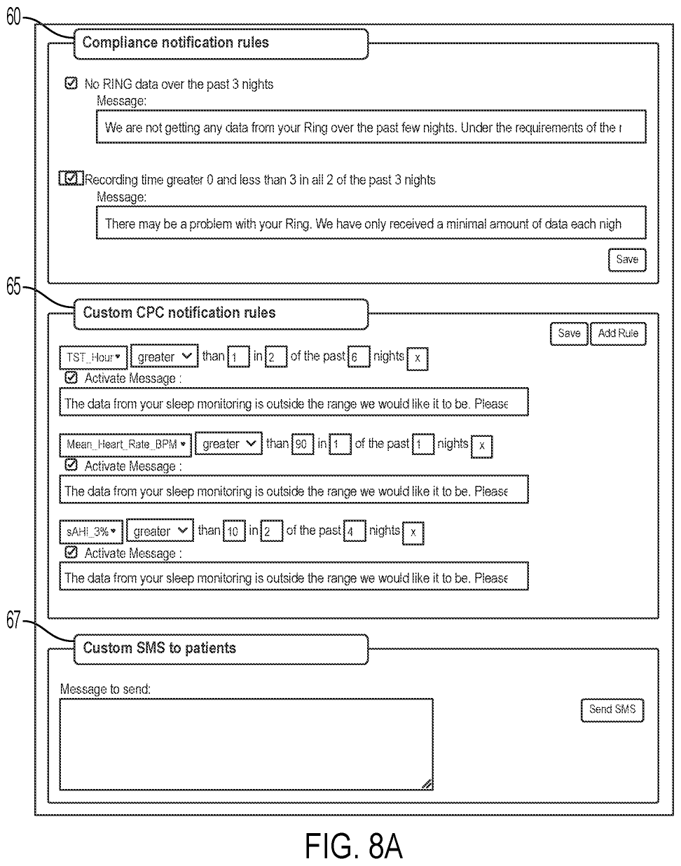

The REST desktop software 10 can also be configured such that each patient has a unique set of thresholds that can be defined by the clinician. For example, in one patient the clinician may not care to identify every night in which their Apnea Hypopnea Index (AHI) is above 15 because relative to their severe OSA, a AHI under 20 would be fine. Whereas in another patient the clinician may wish to be notified if the AHI is greater than 10 for 2 out of 3 consecutive nights. The point here is that the present invention can be tailored to each patient's specific clinical situation as depicted in FIG. 8A, using the custom CPC notification rules 65 where the specific thresholds for different parameters can be set by the clinician. When these thresholds are met then there is a notification that shows up on the dashboard (FIG. 8) with a section that lists those patients on any given day that satisfy that threshold (this section of such listing in FIG. 8 is not shown). Additionally, the system can be set to send a notification via text or email, to the patient informing them that they need to contact the clinician because the results from the monitoring has demonstrated that they have findings of concern. The REST desktop software 10 also provides a means for the patient to view the data collected, as approved by the clinician, from their hand-held device or computer. For example, a patient with OSA who has had poor sleep can view a selectable time to examine a trend of their data. Such as their AHI over the past week, month or three months. Or, they can select to see their total sleep time over the past week, month or three months. The present invention that allows the patients review is under the control of the clinician. In some instances, having access to such data may be disruptive such as in patients with an obsessive-compulsive disorder. The clinician may ask the patient to monitor their sleep to track their insomnia but would prefer the patient not focus on the performance each night, since that can alter the patient's condition. Allowing such control of review is clearly not a feature available by any conventional product.

Referring now to FIGS. 8 and 8A, a “Dashboard” feature of the REST desktop software 10 is illustrated. To manage many patients, data reduction is desirable. The REST desktop software 10 employs an interface that an operator can utilize to bring pertinent information together for review. Also, items of importance that require an action can be easily identified. The Dashboard allows monitoring of certain “vital signs” of a monitored population. Like keeping fingers on a pulse, the Dashboard includes the ability to keep an eye on the clinic population and knows when interventions need to be initiated to keep patients at optimum health. The Dashboard is modifiable such that the operator can set threshold parameters that when met, identifies a patient and lists them on the Dashboard. There are default parameters embedded within the system for a particular patient population. These are all modifiable. The Dashboard allows for daily assessments to establish work action items for the monitoring staff.

As shown in FIG. 8A, the rules for each patient are set up in the notification settings section of the REST desktop software 10. There are several notification parameters that can be set. The first group are referred to as compliance notification rules 60. These correspond to what shows up on the dashboard, for example at the top of FIG. 8A, it states, “No ring data over the past 3 nights” and “Recoding time greater 0 and less than 3 in all 2 of the last 3 nights.” These will show up on the Dashboard, but the middle feature “Custom CPC notification rules” 65 determines if the patient will be notified via text or email message and what message will be sent is placed into these boxes. This is where patient specific custom rules can be set. Any of the parameters monitored by the system can be used for the specific rules. The threshold is set with the rule, such as AHI greater than 15 in 2 out of the past 6 nights. Multiple rules can be set for each patient, such as the example AHI rule above plus, if the patient falls asleep after 1 AM on 2 of the past 4 nights. Then besides the patient showing up on the Dashboard, the patient can also be notified if that feature is selected, and the message sent to the patient via text or email and can be placed into the system. The bottom section “Custom SMS to patients” 67 is a feature where a patient can be sent a text message on demand at any time if the clinical team is trying to contact the patient.

Many of the activities discussed above fall into the category of monitoring events and as such, this provides a billing opportunity (discussed below). For example, FIG. 8 shows how in a single view the Dashboard displays key elements of the patient population being monitored. Elements shown in FIG. 8 are as follows: total patients being monitored; listing of patients meeting some parameter threshold. In this instance the default is set to an sAHI4% greater than 30 on 2 of the past 6 nights. “sAHI” is defined as the SleepImage Apnea Hypopnea Index. Those patients with a sAHI 4% greater than 30 are considered to have severe obstructive sleep apnea, so this lists those patients having severe obstructive sleep apnea in 2 out of the past 6 nights. These patients need to be contacted to assess why their sleep apnea condition has become severe; listing of patients with no CPC ring data over a selected number of nights (default is 3). This allows the ability to list those patients needing to be contacted if there is a malfunction of the ring or patients become non-compliant; listing of patients in which the amount of data is suboptimal, not meeting some threshold; listing of patients in whom there are extended monitoring events being processed (monitoring events are discussed below). These can be listed as, not completed, completed, error (non-sending to the electronic medical record system) or all; listing of patients who have billing that has not been completed by the billing team; and listing of patients needing to be called for the routine monthly communication, as required to comply with CMS billing guidelines. For example, in the case of patients with sleep disorders the Dashboard can display patients that meet specific criteria such as those with an AHI above some threshold, on some number of nights out of the last some number of nights. So, for those patients who meet the criteria, they are considered to have achieved a monitoring event. Monitoring events are processed in stages (initiated, not complete, sent). This tool allows the care giver to keep up with the process of generating monitoring events (as discussed below).

Specific segments of the REST desktop computer program 10 are designed for acquiring information linked to an intervention or some sort of enhanced assessment performed on a patient's monitoring process. This is defined as “Monitoring Events.” Monitoring Events can be documented by a trained staff member who is familiar with the system and can carry out clinically related tasks under the guidance of a licensed clinician. Many CMS billing codes allow tasks to be performed by office staff other than the licensed clinician. This system is designed to capture these Monitoring Events and sending them to a billing system as well as Electronic Medical Record Systems. There are two types of monitoring events, routine and enhanced (or extended). This labeling follows CMS billing rules such that the REST desktop software 10 can capture inputs as deemed necessary by CMS. However, the software can also capture inputs that are of interest, not restricted to a CMS billing guideline. The present invention has scheduling features which sits at the core of the billing engine. The scheduling feature requires a start date and other specific variables, which outlines a recurrent schedule to perform events such as auto generation of reports that can be forwarded to collaborating clinicians or billing prompts sent to the billing team when the recurring period has ended. In such data transfers, specific billing information is automatically placed into the system to reduce staff time. For example, several types if scheduling events can be created, such as billing events, reporting events and other event types. For example, a billing feature is programed to follow the billing rules of insurance companies, such that revenue cycle management can occur within the same system used to monitor the patient. The system will track when medical staff communicate with patients such that billing events are recorded and sent to an electronic medical records system and a billing charge is generated. Such events can only occur in a designated timeline fashion, the rules of which are followed by the REST desktop software 10. The present invention also has a feature for direct voice communication. This is a feature that simplifies compliance with CMS requirements. CMS requires at a minimum, one synchronous communication with a patient each month, via voice as a minimum, in order to allow for billing certain CPT procedure codes. It has been established that text messaging or leaving phone messages does not qualify as meeting this minimum requirement. That is, Medicare (CMS) requires a “real time” synchronous communication, such as a phone call, between the patient and their doctor, or doctors qualified staff member, once a month in order to bill for remote patient monitoring (RPM), or remote therapeutic monitoring (RTM). Doctors then use Medicare billing codes, such as 99457, 99458, 98980 or 98981 (for RPM or RTM). The REST mobile app 20 has a feature where a patient is alerted that they need to have a “check in” with a doctor or qualified staff member using real time synchronous voice communication. The REST mobile app 20 has a VOIP (voice over IP) such that they can press the button and be instantly connected to a live attendant 24 hours a day for the purposes of “checking in” to meet the CMS criteria. This ability for live direct communication has a multitude of applications beyond specifically meeting the CMS criteria. For example, when such calls come into the central operator, it is received with the specific patient automatically pulled, being identified by an identifier linked to the incoming call, and all appropriate notes are pulled up for the operator so that they know what to communicate to the patient regarding the monitoring activity. This feature of the present invention solves the ongoing problem of communicating with patients regarding their treatment or condition. It bypasses standard communication pathways that are customarily used, such as patients calling the main office number and then needing to be routed to a specific person who needs to receive the call, when the patient does not remember what direct number to call or extension to press etc. And also it bypasses the patients standard voice messaging mail or email system.

In summary, the present invention provides several features and advantages not found in todays clinical practice. For example, one is a method for acquiring physiologic data, where a patient puts on device, such as a wearable ring, CPAP mask, or other biosensor or monitor, in some instances this occurs in a time sensitive fashion such as before bed. The patient completes the study period and removes, or replaces, the device. In some instances, the data is to be transmitted when initiated by the patient, in other instances data is automatically transmitted, depending on the type of device measuring the physiologic data. A user interface can capture patient inputs (annotations) that are appropriately synchronized with the physiologic data. The computer software of the present invention synchronizes patient inputs. Different types of inputs require different types of synchronization. For example, some inputs refer to an entire night and others are very time sensitive with significance of the exact time being entered. Some annotated inputs are placed ahead of an event (“tonight I will use a new type of CPAP mask”), and others refer to a point after an event (example—“last night I woke up multiple times and now with a headache”). The user can select on_during the input process if the comment pertains to tonight or last night, which changes the synchronization of the input. The data collected is reduced to combine physiologic data with patient's annotations. The REST desktop software 10 provides elastic zoom to view collected patient data over different time frames. With an ability to expand and select a time frame or period of data capture, with algorithms developed to synchronize patient data and indicate abnormalities or changes, such as those based on cardiopulmonary coupling output metrics. Then the patient or clinician(s) view the data period or element(s) of interest. The clinician uses insight from the data to recognize relationships between treatment changes, other variable factors and physiologic effects. This drives improved medical care decisions.

In another aspect of the present invention, a patient logs into the Assessment Forms (shown in FIG. 4) using an unique identifier and password, and completes a desired form. Or, the patient optionally leaves a note or comment in an available text field. The patient hits a submit button and the patient's response is time stamped in the saved record, which may be viewed in relation to the collection over time. In addition, the patient may log into the Therapy Tracker (see FIGS. 3 and 3A) page 42 of the REST mobile app 20 using an unique identifier and password. The patient then chooses from a series of selectable radio buttons or other touch screen indicator from a menu comprising medication therapy, oral, appliance therapy, CPAP therapy, respiratory therapy, or another type of therapy. Or, the patient optionally leaves a note or comment in an available text field, which is time-stamped to provide synchronized data to collection streams, with an indication of which time period the comment pertains to (for example, “last night” or “tonight”). The patient hits the Submit button and the Patient response is synchronized to the chosen time period of data collection, viewable in elastic time frames chosen by the viewer (for example, user or clinician) on the REST desktop software 10.

Another aspect of the present invention provides synchronized monitoring and treatment adjustment in a patient, where dentists or oral appliance changes can be captured in real time within the same collaborative data stream. This provides a system independent of a specific electronic health record or record system or database. For example, input data is received from the patient and associated devices. Data reduction is performed computationally in the software. Time stamped data is combined graphically to associate a time period or event time with a respective patient input. Graphic output of the data is then viewed by the clinician. In addition, time stamped data is combined graphically to associate a time period or event time with a respective patient input. The software generates an alarm or notation identifying an event has happened or a threshold has been detected. Graphic output of the data is viewed by the medical staff to review, and medical staff contacts the patient to assist with remote monitoring or remote treatment adjustments.

Another aspect of the present invention provides methods of collaboration. For example, a referring clinician can access the grapher displays to view the information of a subject, and can provide clinical information, such as therapy alteration or initiation. In addition, a patient's physiologic event is monitored in a real time, or actual time continuous data stream.

A clinician can monitor real-time data from one prescribed device, such as an dental oral appliance, EEG, cardiopulmonary coupling, or other physiologic parameter in relation to the patient's global sleep data. The clinician can communicate directly with one or more other clinicians caring for the patient within the REST desktop software 10 workspace or display. One or more clinicians can adjust the patient's therapy or treatment based on the patient's physiologic event data at a specific time point or over a time period. The clinician's captured communication is stored as part of the electronic record with both the main monitoring clinician and collaborating clinician's annotations being placed along the same timeline but distinguished by different icons that are distinguished by color and/or shape, as shown in FIG. 5. The event and/or category or time point or time period continues to be tracked in real time during the collaboration of clinicians to monitor the effect of the treatment or therapy alteration, and the software generates summary reports for the clinicians with estimated engagement time and quantified outcomes or changes in patient parameters for a given specified time period. In addition, another embodiment of the present invention may include a statistics or machine learning or artificial intelligence software is used to analyze the changes to treatment in relation to the patient's sleep data and to predict or indicate a health threshold or trajectory. The event pattern and algorithm generates a recommendation for therapy or therapy adjustment for the clinician based on the outcome association or prediction and the clinician orders alteration of the medication, treatment or therapy.

As disclosed above, the present invention will enable a practice to evolve and improve the current standard of care by providing a trusted FDA approved E2E measurement system for continuous measurement-monitoring and intervention. The patient ring-based instrumentation and REST desktop 10 and REST mobile 20 software and coaching aspect offer a complete end to end advanced care system. Allowing for data and rule-based triggering of events (statistically designed to separate common cause from special cause variation) which ultimately separates signal from noise of critical clinical parameters. This plus the additive patient comments enrich the practice and clinician information to develop appropriate intervention (care plan adjustment, drug titration, etc.). The present invention also provides a population health measurement system, that will evolve practice learning and know-how to manage the disease state at a large cohort and individual patient level. This evolving system will allow for the overall quality and cost of care to advance and ultimately improve outcomes. The patient instrumentation and portal level analytics, and subsequent clinical intervention triage will allow for programs of care to advance for risk and complex care management at population and patient level. In addition, the following features are key for regulators (CMS, HHS) and payers in overall advancement of care models and changing incentives and payment models. The cost of care—the present invention will enable less ER utilization, reactive based high-cost site of service visits, episodic management based on early detection, quantification, frequency and magnitude response. The quality of care—the present invention will enable improvement in disease from a proactive perspective. Specifically, data measurement and clinical parameter measurement and intervention resulting in population risk stratification, tiered response design and ultimately patient care. The present invention will also improve outcomes by enabling patient and population baseline characterization, patient care plan design and improvement in clinical outcomes, management of escalated events, reduction of frequency, control and maintenance of improved base line, practice quality and business improvement.

As mentioned above, the REST desktop software 10 and REST mobile app 20 may be implemented by appropriately programmed general purpose computers and computing devices. Typically a processor (i.e., a microprocessor) will receive instructions from a memory or like device, and execute those instructions, thereby performing a process defined by those instructions. Further, programs that implement such methods and algorithms may be stored and transmitted using a variety of known media. A provided embodiment is directed to a method of operating a portable electronic device 25. For example, the REST mobile app 20 may be employed on a cell phone, electronic tablet or other portable electronic device 25 that contains a microphone, communication circuits, a processor, and a display.

The REST desktop software 10 and REST mobile app 20 may employ a network 35 that may include any one or both of a voice call and data communication using a public telephone network, in addition to communication using a wired or wireless TCP/IP protocol including Gigabit Ethernet, 4G, and 5G. For example, a cellular communication network area is divided into cells, each containing network equipment that includes a Base Station Subsystem (BSS). A typical BSS will include a Base Transceiver Station (BTS) which contains the equipment for transmitting and receiving radio waves. Equipment can include transmitters, receivers, antennas, amplifiers, encryption and decryption equipment and equipment configured to communicate with a Base Station Controller (BTS). As envisioned, the portable electronic devices 25 (i.e., cell phones, tablets, laptops or other electronic devices) are in communication with network equipment 35. In this depiction the portable electronic device 25 is illustrated as a smartphone or cell phone. The progression of cellular technology through 2G, 2.5G, 3G, 4G, 5G and future systems has added additional functionality to the network 35 and allows it to support a wider range of portable electronic devices 25. As shown in FIG. 1, portable electronic equipment 25 is in communication with network equipment 35 that is in communication with the voice Public Switched Telephone Network (PSTN). In addition, the network 35 may include a Data IP network. The network 35 may also include a 3G cellular network responsible for allowing the portable electronic device 25 to access the Radio Network Subsystem (RNS). A 3G network communicates to both data (IP) and to Voice (PSTN) Network.

In addition, the network 35 may include a 4^{th}, 5^{th} or next Generation network architecture. For example, a 4G cellular access network consists of network equipment and access points. It will be appreciated by those of skill in the art that communication in a 2G network consists of circuit switched communication whereas in 4G, the communication with the network is all packet switched. Further, many portable electronic devices 25 are backwards compatible with 2G, 3G, 4G, and 5G networks. Indeed, the invention is not limited to any particular network equipment 35.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article. The functionality and/or the features of a device may be alternatively embodied by one or more other devices which are not explicitly described as having such functionality/features. Thus, other embodiments of the present invention need not include the electronic device 25 itself.

The present invention may be implemented on a computer-readable medium. The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read. Moreover, the present invention may also be downloaded as a computer program product, wherein the program may be transferred from a remote computer to a requesting computer by way of data signals embodied in a carrier wave or other propagation medium via a communication link (e.g., a wired/wireless modem or network connection).

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G, 4G, 5G and ultrawideband.

The following discussion is intended to provide a brief, general description of a suitable computing environment in which the invention may be implemented. Although not required, the invention may be described in the general context of computer-executable instructions, such as program modules, being executed by a personal computer or portable electronic device, such as a cell phone or electronic tablet. Generally, program modules include routines, programs, objects, components, data structures, etc. that perform particular tasks or implement particular abstract data types. Moreover, those skilled in the art will appreciate that the invention may be practiced with other computer system configurations, including hand-held devices, multiprocessor systems, microprocessor-based or programmable consumer electronics, network PCs, minicomputers, mainframe computers, and the like. The invention may also be practiced in distributed computing environments where tasks are performed by remote processing devices that are linked through a communications network. In a distributed computing environment, program modules may be located on both local and remote memory storage devices.

An exemplary system for implementing the invention includes a general-purpose computing device in the form of a conventional personal computer, including a processing unit, a system memory, and a system bus that couples various system components including the system memory to the processing unit. The system bus may be any of several types of bus structures including a memory bus or memory controller, a peripheral bus, and a local bus using any of a variety of bus architectures. The system memory includes read only memory (ROM) and random access memory (RAM). A basic input/output system (BIOS), containing the basic routines that helps to transfer information between elements within the personal computer, such as during start-up, is stored in ROM.

The personal computer also includes a computer hard disk drive (HDD), for flash memory that comprises a non-volatile data storage device. Non-volatile refers to storage devices that maintain stored data when turned off. All computers need a storage device, and HDDs are just one example of a type of storage device.

The drives and their associated computer-readable media provide nonvolatile storage of computer readable instructions, data structures, program modules and other data for the personal computer. Although the exemplary environment described herein employs a hard disk, it should be appreciated by those skilled in the art that other types of computer readable media which can store data that is accessible by a computer, such as magnetic cassettes, flash memory cards, digital video disks, Bernoulli cartridges, random access memories (RAMs), read only memories (ROM), and the like, may also be used in the exemplary operating environment.

A number of program modules may be stored on the hard disk, ROM or RAM, including an operating system, one or more application programs, other program modules, and program data. A user may enter commands and information into the personal computer through input devices such as a keyboard and pointing device. These and other input devices are often connected to the processing unit through a serial port interface that is coupled to the system bus, but may be connected by other interfaces, such as a parallel port, game port or a universal serial bus (USB). A monitor or other type of display device is also connected to the system bus via an interface, such as a video adapter. In addition to the monitor, personal computers typically include other peripheral output devices (not shown), such as speakers and printers.

The personal computer may operate in a networked environment using logical connections to one or more remote computers, such as a remote computer. The remote computer may be another personal computer, a server, a router, a network PC, a peer device or other common network node, and typically includes many or all of the elements described above relative to the personal computer. The logical connections include a local area network (LAN) and a wide area network (WAN). Such networking environments are commonplace in offices, enterprise-wide computer networks, intranets, and Internet.

When used in a LAN networking environment, the personal computer is connected to the local network through a network interface or adapter. When used in a WAN networking environment, the personal computer typically includes a modem or other means for establishing communications over the wide area network, such as the Internet. The modem, which may be internal or external, is connected to the system bus via the serial port interface. In a networked environment, program modules depicted relative to the personal computer, or portions thereof, may be stored in the remote memory storage device. It will be appreciated that the network connections are exemplary and other means of establishing a communications link between the computers may be used, including wireless methods, such as WiFi, Bluetooth or other wireless protocols.

As will be appreciated by one of skill in the art, embodiments of the present invention may be provided as methods, systems, or computer program products. Accordingly, the present invention may take the form of an entirely hardware embodiment, an entirely software embodiment or an embodiment combining software and hardware aspects. Furthermore, the present invention may take the form of a computer program product which is embodied on one or more computer-usable storage media (including, but not limited to, disk storage, CD-ROM, optical storage, integrated circuits and so forth) having computer-usable program code embodied therein.

These computer program instructions may also be stored in a computer-readable memory that can direct a computer or other programmable data processing apparatus to function in a particular manner, such that the instructions stored in the computer-readable memory produce an article of manufacture including instruction means which implement the functions discussed above in this specification.

With reference to the above-disclosed invention, it is noted that devices that are in communication with each other need not be in continuous communication with each other, unless expressly specified otherwise. In addition, devices that are in communication with each other may communicate directly or indirectly through one or more intermediaries. For example, communication between the REST desktop software 10 and the REST mobile app 20.

A description of an embodiment with several components in communication with each other does not imply that all such components are required. On the contrary a variety of optional components are described to illustrate the wide variety of possible embodiments of the present invention.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

Thus, it is seen that a monitoring system for humans is provided. One skilled in the art will appreciate that the present invention can be practiced by other than the above-described embodiments, which are presented in this description for purposes of illustration and not of limitation. The specification and drawings are not intended to limit the exclusionary scope of this patent document. It is noted that various equivalents for the particular embodiments discussed in this description may practice the invention as well. That is, while the present invention has been described in conjunction with specific embodiments, it is evident that many alternatives, modifications, permutations and variations will become apparent to those of ordinary skill in the art in light of the foregoing description. Accordingly, it is intended that the present invention embrace all such alternatives, modifications and variations. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order.

What is claimed is:

1. A method for acquiring physiologic data from a patient, the method comprising the steps of:
attaching a device to the patient;
establishing a time period for the device to obtain data from the patient;
sending data from the device to a portable electronic device;
receiving a plurality of inputs from the patient related to the time period;
correlating each of the plurality of inputs with a specific moment during the time period; and
determining a relationship between a patient treatment and the plurality of inputs received during the time period.

2. The method of claim 1, where the device is selected from a group consisting of: a dental appliance; a ring sized to be worn on a finger of the patient; a watch sized to fit a wrist of the patient; a continuous positive airway pressure device; a blood pressure monitor device; a strap sized to fit a wrist of the patient; a strap sized to fit a chest of the patient; a cardiopulmonary sensor; and a combination of two or more thereof.

3. The method of claim 1, where the portable electronic device is selected from a group consisting of: a cellular phone; an electronic tablet; a portable computer; a laptop computer; and a combination of two or more thereof.

4. The method of claim 1, where the time period can range from a fraction of a second to a multiplicity of days.

5. The method of claim 1, where the data sent from the device is selected from a group consisting of: a fragmented sleep period; a consolidated sleep period; a cessation of respiration; a grinding of teeth; a non-sleep period; a leg movement; and a combination of two or more thereof.

6. The method of claim 1, where each of the plurality of inputs from the patient is selected from a group consisting of: an oxygen saturation level, a heart rate; a heart rate pattern, a respiration rate; a respiration pattern, a glucose level; a pattern of glucose levels, a change in an Apnea-Hypopnea Index, an electroencephalogram; an electroencephalogram pattern; an electromyography signal; an electromyography pattern; a biomarker signal received from an implanted device; and a combination of two or more thereof.

7. The method of claim 1, where the patient treatment is selected from a group consisting of: at least one medication; a continuous positive airway pressure device; a hypoglossal nerve stimulator device; an oral appliance; a combination of two or more thereof.

8. A method of operating a communication network, the communication network comprising a plurality of network equipment, the method comprising:
monitoring a physiologic event of a patient in an actual time;
obtaining a sleep data of the patient in an actual time;
displaying the sleep data and the physiologic event of the patient simultaneously on the plurality of network equipment;
transmitting in actual time the sleep data and the physiologic event to the communication network so that at least two remotely located care givers can view the sleep data and the physiologic event on the plurality of network equipment in actual time; and
adjusting a therapy for the patient based on the physiologic event.

9. The method of claim 8, further comprising the steps of:
displaying the physiologic event and the sleep data in a linear timeline;
displaying a first icon adjacent to the linear timeline representing the therapy adjustment recommended by a first care giver thereby establishing a visual relationship with the physiologic event, where the first icon can be selected and an annotation associated with the first icon can be viewed; and
displaying a second icon adjacent to the linear timeline representing an assessment of the therapy adjustment by a second care giver, where the second icon can be selected and an annotation associated with the second icon can be viewed.

10. The method of claim 8, further comprising the step of:
displaying in an actual time the sleep data after the therapy to determine an effect of the therapy.

11. The method of claim 8, further comprising the step of:
monitoring a physiologic event of a second patient in an actual time;
creating a unique identifier for each patient; and
generating a report for the care givers, the report comprising any changes in a parameter of each patient, with each patent identified by their unique identifier.

12. The method of claim 11, wherein the parameter of the patient is selected from a group consisting of: an oxygen saturation level, a heart rate; a heart rate pattern, a respiration rate; a respiration pattern, a glucose level; a pattern of glucose levels, a change in an Apnea-Hypopnea Index, an electroencephalogram; an electroencephalogram pattern; an electromyography signal; an electromyography pattern; a biomarker signal received from an implanted device; and a combination of two or more thereof.

13. The method of claim 8, where the monitoring of the physiologic event of the patient includes a continuous data stream of the physiologic event that is transmitted over the communication network.

14. The method of claim 8, further comprising the step of:
Transmitting data from the physiologic event from the patient to the care givers over the communication network using an encryption code.

15. The method of claim 8, further comprising the step of:
transmitting the physiologic event automatically to the care givers over the communication network.

16. A method of operating a communication network, the communication network comprising a plurality of network equipment, the method comprising:
monitoring a physiologic parameter of a patient;
displaying the physiologic parameter on the plurality of network equipment in the form of a linear timeline;
transmitting the physiologic parameter and timeline through the communication network so that a plurality of remotely located care givers can view the physiologic parameter and timeline on the plurality of network equipment;
adjusting a therapy for the patient based on the physiologic parameter;
displaying a first icon adjacent to the linear timeline representing a therapy adjustment recommended by a first care giver thereby establishing a visual relationship with the physiologic parameter, where the first icon can be selected and an annotation associated with the first icon can be viewed; and
displaying a second icon adjacent to the linear timeline representing an assessment of the therapy adjustment by a second care giver thereby establishing a visual relationship with the physiologic parameter, where the second icon can be selected and an annotation associated with the second icon can be viewed.

17. The method of claim 16, where the physiologic parameter is selected from a group consisting of: a blood pressure; a blood sugar; a heart rate; a respiration rate; a sleep condition; and a combination of two or more thereof.

18. The method of claim 16, further comprising the steps of:
monitoring a second physiologic parameter of a patient in an actual time; and
displaying the second physiologic parameter on the plurality of network equipment;
transmitting in actual time both physiologic parameters to the communication network so that a second remotely located care giver can view the physiologic parameter on the plurality of network equipment.

19. The method of claim 16, further comprising the steps of:
communicating synchronously to at least two care givers through the network equipment;
collaborating using the network equipment to recommend a therapy modification;
assessing a status of the patient after the therapy modification;
recording the therapy modification so that the at least two care givers can view the therapy modification; and
continuing the collaboration using the network equipment until the therapy modification shows a desired effect on the patent.

20. The method of claim 19, further comprising the steps of:
displaying the therapy modification of a population of patients by one or more care givers; and
determining a relationship between the therapy modification and a change in a patient condition by viewing the therapy modification of the population.
